(19) United States
(12) Patent Application Publication
(10) Pub. No.: US 2014/0094661 A1
(43) Pub. Date: Apr. 3, 2014

(54) CLINICAL INFORMATION DISPLAY APPARATUS, METHOD, AND PROGRAM

(71) Applicant: FUJIFILM Corporation, Tokyo (JP)

(72) Inventors: Meiji ITOH, Ashigarakami-gun (JP); Yasunori Ohta, Ashigarakami-gun (JP); Akira Oosawa, Ashigarakami-gun (JP); Shoji Kanada, Ashigarakami-gun (JP)

(73) Assignee: FUJIFILM Corporation, Tokyo (JP)

(21) Appl. No.: 14/020,697

(22) Filed: Sep. 6, 2013

(30) Foreign Application Priority Data
Sep. 28, 2012 (JP) 2012-216623

(51) Int. Cl. A61B 5/00

(52) U.S. Cl. CPC A61B 5/743; A61B 5/746; A61B 5/7282
USPC 600/301

(57) ABSTRACT

A clinical information display apparatus in which a plurality of clinical information items is classified into display and non-display targets and clinical information corresponding to a clinical information item classified as the display target is displayed on a display unit, including an anomaly detection unit for detecting an anomaly in clinical information corresponding to a clinical information item classified as the non-display target, and a notification unit for, when an anomaly is detected by the anomaly detection unit, making a notification to that effect.

### BACKGROUND OF THE INVENTION

1. Field of the Invention

The present invention relates to a clinical information display apparatus, method, and program for displaying important clinical information required for diagnosing a possible disease of a patient from a plurality of pieces of clinical information of the patient.

2. Description of the Related Art

In the field of medicine, various types of clinical information obtained from a patient are displayed on a display device as reference information for diagnosing a disease name and determining treatment policy.

For example, Japanese Unexamined Patent Publication No. 2010-061389 (Patent Document 1) proposes a method that allows the user, such as a doctor or the like, to efficiently refer to clinical information of interest when a patient is suspected to have a particular disease by restricting the display target only to some of the pieces of clinical information required for the diagnosis of the patient, i.e., by not displaying the remaining pieces of clinical information which are not necessary for the diagnosis of the patient, because clinical information items have diversified with the recent advancement of medical technologies and there are many cases in which a large number of pieces of clinical information are present for one patient and it is unrealistic to display all of the pieces of clinical information obtained from the patient. Further, Japanese Unexamined Patent Publication No. 2001-133293 (Patent Document 2) discloses a technology that, when displaying a plurality of examination data (clinical information), displays each of the examination data in a visually distinguishable manner as to whether the value thereof is a normal value or an abnormal value outside of a normal value range.

### SUMMARY OF THE INVENTION

The conventional method that restricts the display target only to some of the pieces of clinical information required for the diagnosis of a specific patient is effective for improving the efficiency of reference to clinical information of interest, but other pieces of clinical information not displayed as a result of the restriction may possibly be overlooked even though they sometimes give a sign of complications or other diseases.

In view of the circumstances described above, it is an object of the present invention to provide a clinical information display apparatus, method, and program which, when a plurality of clinical information items is classified into display and non-display targets, and clinical information corresponding to a display target clinical information item is displayed, alerts the user of the presence of non-display clinical information that may give a sign of complications or other diseases and prevent overlooking.

A clinical information display apparatus of the present invention is a clinical information display apparatus in which a plurality of clinical information items is classified into display and non-display targets and clinical information corresponding to a clinical information item classified as the display target is displayed on a display unit, the apparatus including:

an anomaly detection unit for detecting an anomaly in clinical information corresponding to a clinical information item classified as the non-display target; and

a notification unit for, when an anomaly is detected by the anomaly detection unit, making a notification to that effect.

Here, the notification unit may make a notification that an anomaly has been detected by displaying a message to that effect on the display unit or issuing a warning sound.

The term “clinical information” as used herein refers to information of a patient obtained for diagnosis which may be obtained through examination of a patient, declaration from the patient, and the like. Specific examples include basic patient information, such as blood pressure, pulsation, age, gender, body weight, body temperature, number of respirations, and the like, major complaint, history of life, medical history, family history, various examination result data, various image findings and image characteristic amounts, medication/treatment contents, treatment results, gene information, severity level, and the like.

The term “clinical information item” as used herein refers to an item representing a type of clinical information and, for example, a blood pressure, pulsation, age, gender, body weight, body temperature, number of respirations, or the like may be a clinical information item with respect to basic patient information, an examination item may be a clinical information item with respect to blood examination, a modality type may be a clinical information item with respect to image inspection, and a type of medicine may be a clinical information item with respect to medication information.

In the clinical information display apparatus of the present invention described above, the notification unit may be constructed to display, when an anomaly is detected by the anomaly detection unit, information with respect to the clinical information in which the anomaly has been detected on the display unit in addition to making the notification.

Specific examples of the “information with respect to the clinical information” include an item name of the clinical information, clinical information itself, information of a disease associated with a clinical information item, and the like.

The clinical information display apparatus of the present invention described above may include a correspondence relationship storage unit storing a correspondence relationship between each clinical information item and a disease associated with the item, and the notification unit may be constructed to identify, when an anomaly is detected by the anomaly detection unit, a disease corresponding to the clinical information item of the clinical information in which the anomaly has been detected from the correspondence relationship stored in the correspondence relationship storage unit and to display information with respect to the disease (e.g., disease name) on the display unit in addition to making the notification.

The clinical information display apparatus of the present invention described above may include a mode setting unit for setting a restriction mode that restricts clinical information items for which the notification is made by the notification unit, and the notification unit may be constructed to make, when the restriction mode is set by the restriction mode setting unit, the notification only for clinical information items not restricted.

Here the restriction mode may be a mode that restricts the notification for all clinical information items. Otherwise, the restriction mode may be a mode that restricts the notification only for some of the clinical information items having a low level of importance and the notification is made only for clinical information items having a high level of importance.

The clinical information display apparatus of the present invention described above may further include an input receiving unit for receiving a user input for selecting any disease whose information is displayed on the display screen by the notification unit, and a display control unit for, when a user input for selecting a disease is received by the input receiving unit, switching the display screen of the display unit to a display screen in which clinical information corresponding to the clinical information item associated in advance with the selected disease is displayed.

In the clinical information display apparatus of the present invention described above, the anomaly detection unit may be constructed to detect an anomaly in clinical information constituted by a series of numerical data based on a slope of a graph generated by the clinical information or to detect an anomaly in clinical information constituted by a series of numerical data obtained over a plurality of times based on whether or not the state in which the numerical data constituting the clinical information are outside of a predetermined criterion value range has continued not less than a predetermined period of time.

A clinical information display method of the present invention is a clinical information display method performed by a clinical information display apparatus which includes an anomaly detection unit and a notification unit, wherein, when a plurality of clinical information items is classified into display and non-display targets and clinical information corresponding to a clinical information item classified as the display target is displayed on a display unit, the anomaly detection unit detects an anomaly in clinical information corresponding to a clinical information item classified as the non-display target and when an anomaly is detected, the notification unit makes a notification to that effect.

In the clinical information display method described above, when an anomaly is detected, the notification unit may display information with respect to the clinical information in which the anomaly has been detected on the display unit in addition to making the notification.

A clinical information display program of the present invention is a clinical information display program for causing a computer to function as a clinical information display apparatus in which a plurality of clinical information items is classified into display and non-display targets and clinical information corresponding to a clinical information item classified as the display target is displayed on a display unit, wherein the clinical information display apparatus includes:

an anomaly detection unit for detecting an anomaly in clinical information corresponding to a clinical information item classified as the non-display target; and

a notification unit for, when an anomaly is detected by the anomaly detection unit, making a notification to that effect.

According to the clinical information display apparatus, method, and program of the present invention, when a plurality of clinical information items is classified into display and non-display targets and clinical information corresponding to a clinical information item classified as the display target is displayed on a display unit, an anomaly in clinical information corresponding to a clinical information item classified as the non-display target is detected and when an anomaly is detected, a notification to that effect is made. This may alert the user of the presence of non-display clinical information that may give a sign of complications or other diseases and prevent overlooking.

In the clinical information display apparatus of the present invention described above, if the notification unit is constructed to display, when an anomaly is detected by the anomaly detection, information with respect to the clinical information in which the anomaly has been detected on the display unit in addition to making the notification, the user may know from the display in which clinical information the anomaly has been detected and take an appropriate action, such as diagnosing and the like, as required.

If the clinical information display apparatus of the present invention described above includes a correspondence relationship storage unit storing a correspondence relationship between each clinical information item and a disease associated with the item, and the notification unit is constructed to identify, when an anomaly is detected by the anomaly detection unit, a disease corresponding to the clinical information item of the clinical information in which the anomaly has been detected from the correspondence relationship stored in the correspondence relationship storage unit and to display information with respect to the disease on the display unit in addition to making the notification, the user may know the disease with respect to the clinical information in which the anomaly has been detected from the display and take an appropriate action, such as diagnosing and the like, as required.

If the clinical information display apparatus of the present invention described above includes a mode setting unit for setting a restriction mode that restricts clinical information items for which the notification is made by the notification unit, and the notification unit is constructed to make, when the restriction mode is set by the restriction mode setting unit, the notification only for clinical information items not restricted, for example, clinical information having a low level of importance for the user is not notified whereby work efficiency degradation due to responding to unnecessary notification may be prevented.

If the clinical information display apparatus of the present invention described above further includes an input receiving unit for receiving a user input for selecting any disease whose information is displayed on the display screen by the notification unit, and a display control unit for, when a user input for selecting a disease is received by the input receiving unit, switching the display screen of the display unit to a display screen in which clinical information corresponding to the clinical information item associated in advance with the selected disease is displayed, the user may know clinical information necessary for the diagnosis of the disease with respect to the clinical information in which the anomaly has been detected from the display and take an appropriate action, such as diagnosing and the like, as required.

### BRIEF DESCRIPTION OF THE DRAWINGS

FIG. 1 schematically illustrates a configuration of a clinical information system to which a clinical information display apparatus of the present invention is applied.

FIG. 2 illustrates an example display filter that predetermines the correspondence relationship between the clinical information item and disease.

FIG. 3 illustrates an example display screen displayed by the display control unit 24.

FIG. 4 illustrates another example display screen displayed by the display control unit 24.

### DESCRIPTION OF THE PREFERRED EMBODIMENTS

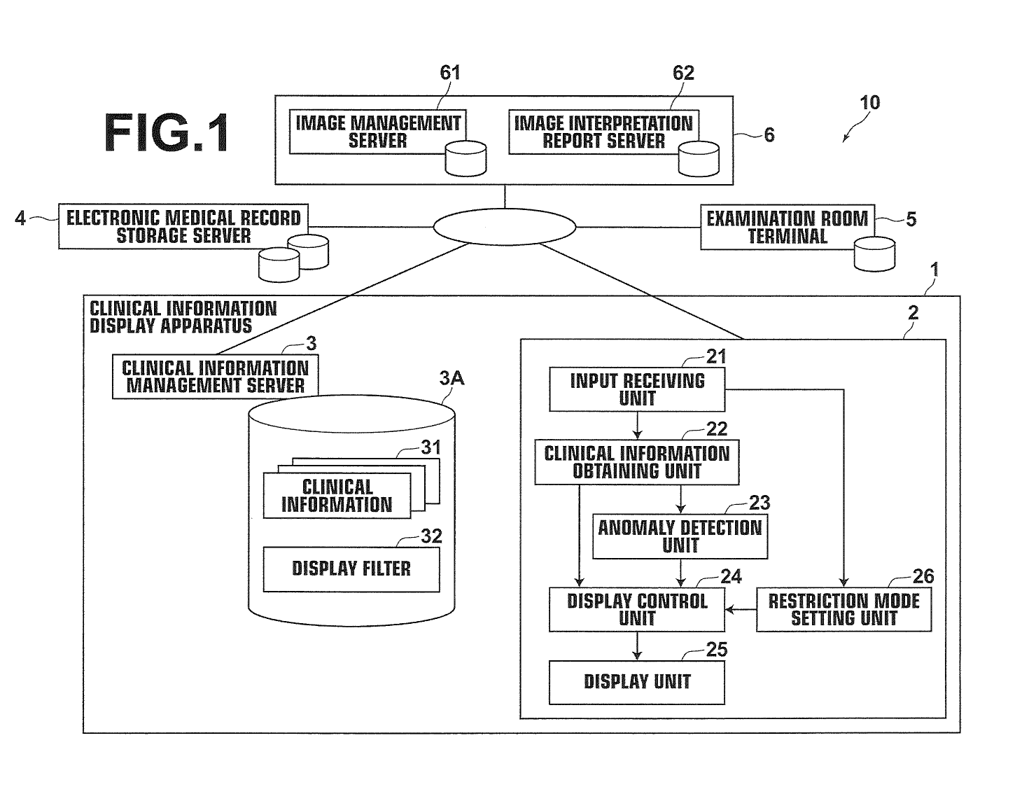

Hereinafter, an embodiment of the clinical information display apparatus, method, and program of the present invention will be described with reference to the accompanying drawings. FIG. 1 schematically illustrates a configuration of a clinical information system 10 to which a clinical information display apparatus according to an embodiment of the present invention is applied. As illustrated in FIG. 1, the clinical information system 10 includes a diagnosis/treatment department terminal 2, a clinical information management server 3, an electronic medical record storage server 4, an examination room terminal 5, and image diagnosis system 6 which are communicatively linked via a network 7. The clinical information apparatus 1 according to the present embodiment is constituted by the clinical information management server 3 and the diagnosis/treatment department terminal 2.

The electronic medical record storage server 4 is a computer that includes an electronic medical record database in which electronic medical records are stored and operates/manages the electronic medical record database. The examination room terminal 5 is a computer provided in an examination room and used for entering results of examinations performed in the examination room according to the examination order entered from each of the diagnosis/treatment department terminal 2. The computer includes software for viewing an examination order and entering examination results, in addition to standard software, such as an operation system or the like. The information of results of an examination performed in each examination room is entered from the examination room terminal 5 in association with the examination order or a patient ID. Further, the examination room terminal 5 includes an examination result database for storing the entered information of results of the examination.

The image diagnosis system 6 includes an image management server 61 having an image database storing therein image data obtained by image capturing with modalities such as CT, MRI, and the like and an image interpretation report server 62 having an image interpretation report database storing therein an image interpretation report which includes image interpretation results of the image obtained by the image capturing communicatively linked to each other via the network 7. The image diagnosis medical workstation includes various types of image analysis software and is configured so as to be able to perform various types of image analysis processing according to the purpose and target of the diagnosis.

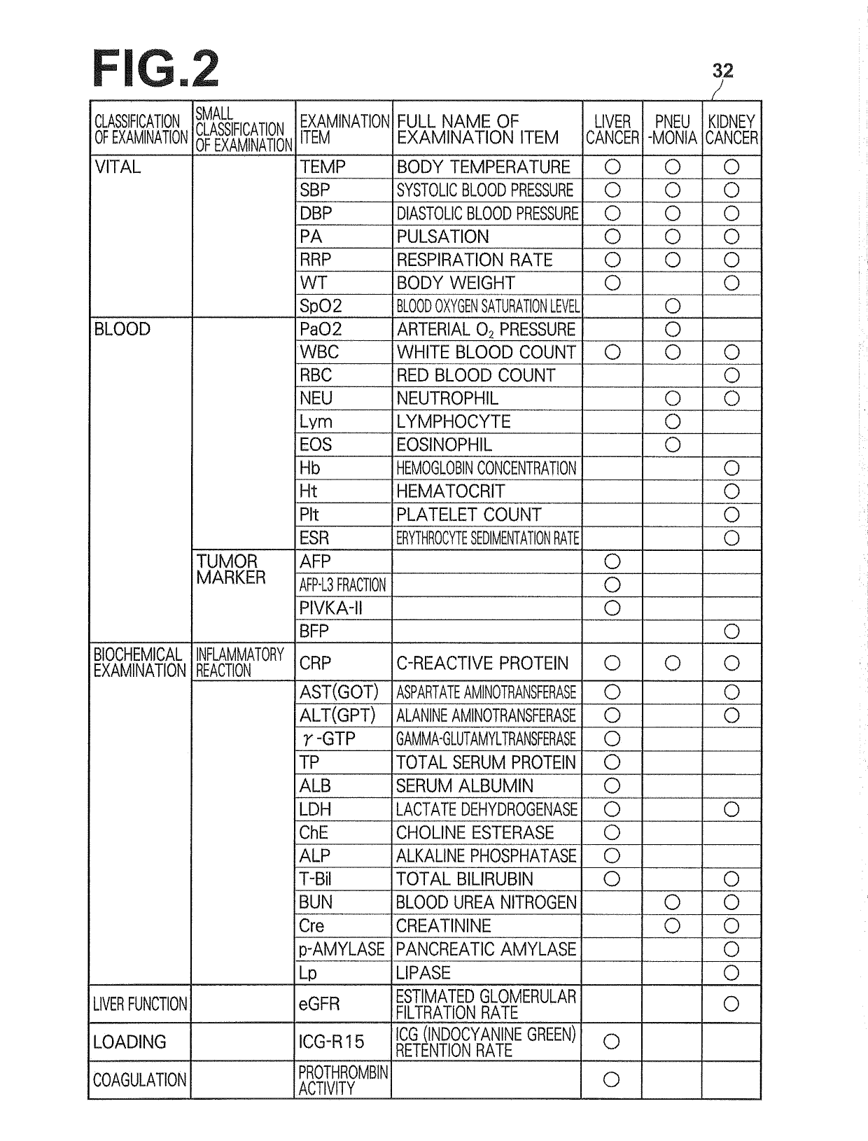

The clinical information management server 3 is a computer that obtains a variety of clinical information of patients from the electronic medical record database of the electronic medical record storage server 4, examination result database of the examination room terminal 5, image database of the image management server 61, and image interpretation report database of the image interpretation report server 62 to build a clinical information database 3A and operates/manages the clinical information database 3A so built. The clinical information database 3A stores a display filter 32 in which an item representing a type of clinical information (clinical information item) and a disease corresponding to the clinical information item are associated with each other in addition to clinical information 31 obtained for patients. An example of display filter 32 is shown in FIG. 2. Note that the display filter 32 corresponds to the “correspondence relationship between each clinical information item and a disease associated with the clinical information item” of the present invention.

The diagnosis/treatment department terminal 2 is a computer used by a doctor of the diagnosis/treatment department or the like for viewing clinical information of a patient, entering an examination order, and the like, and includes a display unit 25, such as a display, and an input receiving unit 21, such as a keyboard and a mouse. The diagnosis/treatment department terminal 2 includes application software (clinical information display program) for realizing clinical information display processing according to the present embodiment in addition to the standard software, such as an operating system. The computer of the diagnosis/treatment department terminal 2 is controlled by the clinical information display program and functions as a clinical information obtaining unit 22, an anomaly detection unit 23, a display control unit 24, and a restriction mode setting unit 26.

The clinical information obtaining unit 22 receives a user input through the input receiving unit 21 to obtain a patient ID of a display target patient and a possible disease of the display target patient, and obtains a piece of clinical information 31 and a display filter 32 of the display target patient from the clinical information database 3A of the clinical information management server 3. Then, based on the display filter 32, the clinical information obtaining unit 22 classifies the clinical information into clinical information corresponding to the disease of the patient and other kinds of clinical information, and outputs the clinical information corresponding to the disease of the patient to the display control unit 24 as the display target on the display unit 25 and the other kinds of clinical information of non-display target to the anomaly detection unit 23.

For example, in the case where pneumonia is specified as the possible disease of the display target patient by the user input received by the input receiving unit 21, the clinical information obtaining unit 22 extracts clinical information corresponding to clinical information items, such as TEMP (body temperature), SBP (systolic blood pressure), DBP (diastolic blood pressure), PA (pulsation), RRP (respiratory rate), SpO2 (blood oxygen saturation level), and the like, corresponding to pneumonia as the display target clinical information from the display filter 32 shown in FIG. 2 and outputs the extracted information to the display control unit 24 and other kinds of clinical information to the anomaly detection unit 23.

The anomaly detection unit 23 detects an anomaly in the non-display target clinical information inputted from the clinical information obtaining unit 22 and, if an anomaly is detected, outputs a signal to that effect to the display control unit. The anomaly detection unit 23 further has a function to output information of clinical information in which an anomaly has been detected and information of a disease corresponding to the clinical information item of the clinical information in which an anomaly has been detected, in addition to the signal that an anomaly has been detected.

Basically, the anomaly detection unit 23 determines an anomaly based on whether or not the target clinical information for anomaly judgment falls outside of a predetermined criterion value range (normal value). Here, the judgment criterion for anomaly may be set or changed appropriately based on the history of the clinical information of the target patient.

In the case where the clinical information is constituted by a series of numerical data that can be represented as a graph, the anomaly detection unit 23 may detect an anomaly based on a slope of a graph generated by the clinical information. For example, if the numerical data of the clinical information are on the increase or decrease towards the anomaly direction with a slope that falls outside of a predetermined criterion value range (slope greater that or equal to a predetermined value or slope less than or equal to the predetermined value), the clinical information item can be determined to be an anomaly.

Further, the anomaly detection unit 23 may detect an anomaly in clinical information constituted by a series of numerical data obtained over a plurality of times based on whether or not the state in which the numerical data constituting the clinical information are outside of a predetermined criterion value range has continued more than or equal to a predetermined period of time. For example, if the state in which the numerical data of the clinical information are outside of a predetermined criterion value range has continued for a long time (e.g., three months or more), the clinical information item can be determined to be an anomaly.

With respect to a clinical information item in which an anomaly can be determined through a combination with the other clinical information item by the nature thereof, the anomaly detection unit 23 may determined the presence or absence of an anomaly by referring to the clinical information corresponding to the other clinical information item. For example, in the case where clinical information of all of specific clinical information items is outside of the criterion value range, the clinical information of these items can be determined to be anomalies. A specific example is that if clinical information of BUN (blood urea nitrogen), Cr (creatinine), and SpO2 (blood oxygen saturation level) is outside of a predetermined criterion value range set to each of the clinical information items, dehydration symptoms caused by pneumonia can be said to be observed, so that these clinical information items are determined to be anomalies in such a case.

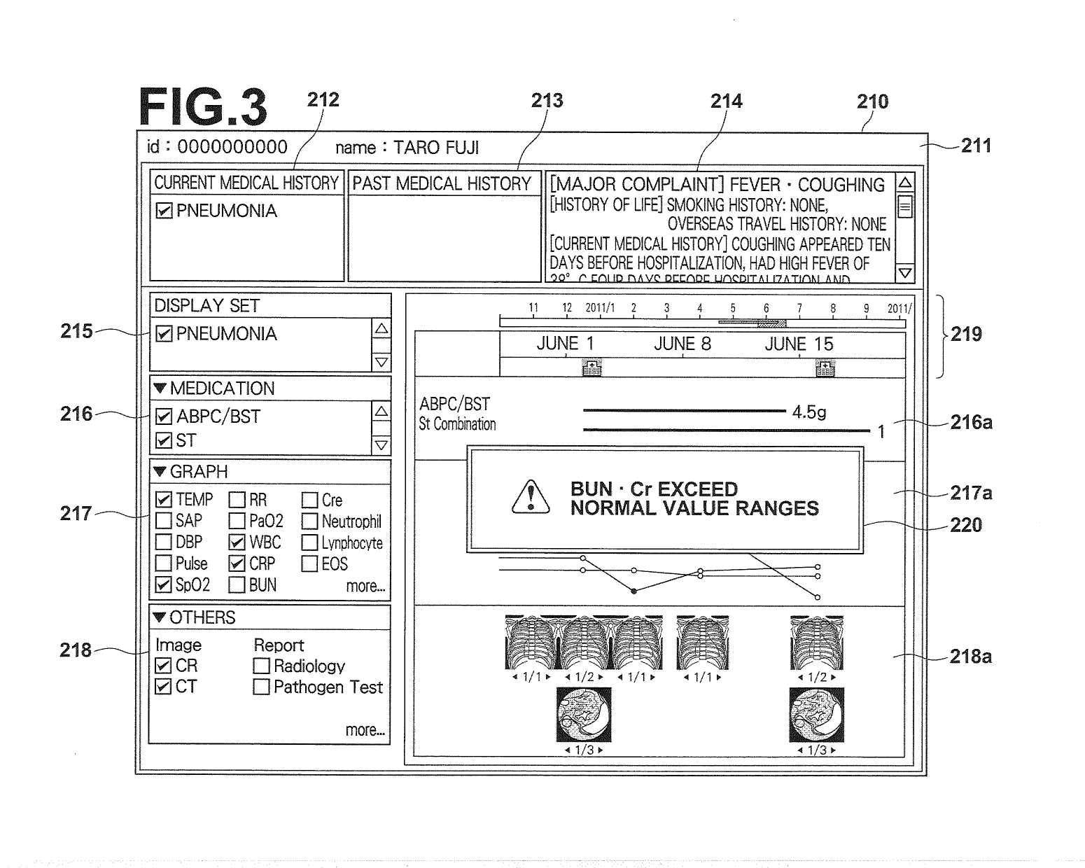

The display control unit 24 displays the display target clinical information inputted from the clinical information obtaining unit 22 on the display unit 25 according to a given display option in which a position and a method for displaying clinical information are preset. Here, if an anomaly is detected in non-display clinical information by the anomaly detection unit 23 and an input to that effect has been received from the anomaly detection unit 23, the display control unit 24 further displays a warning informing that an anomaly is detected in non-display clinical information. In the case where, in addition to a signal indicating that an anomaly has been detected, information with respect to the clinical information in which the anomaly is detected and information with respect to a disease corresponding to the clinical information item of the clinical information in which the anomaly is detected are further inputted from the anomaly detection unit 23 based on a preset given anomaly detection option, the display control unit 24 displays a warning that includes these information. An example of display screen displayed by the display control unit 24 is shown in FIG. 3. In the display screen of FIG. 3, a warning popup 220 notifying that anomalies are detected in BUN•Cr, which are non-display information, is displayed on a display window 210 in which clinical information corresponding to pneumonia specified as a possible disease of a display target patient named Taro Fuji is integrally displayed.

In the display window 210 of FIG. 3, a patient ID and a patient name are displayed on the upper display area 211, and a current medical history, a past medical history, and a medical record are displayed respectively in the display areas 212, 213, and 214 located immediately below the display area 211. Further, the entire display area below the display areas described above is divided into two display areas, and information for identifying a display target clinical information item is displayed in the display area on the left while clinical information corresponding to the display target clinical information item is displayed in the display area on the right. The display areas 215, 216, 217, and 218 on the left display a disease (pneumonia) specified by the user, types of medicines corresponding to the disease, examination items corresponding to the disease, and other display target items respectively. The display areas 219, 216a, 217a, and 218a on the right display a display period of clinical information displayed in the display areas on the lower side, medication information, a graph of examination data, and other display target information respectively.

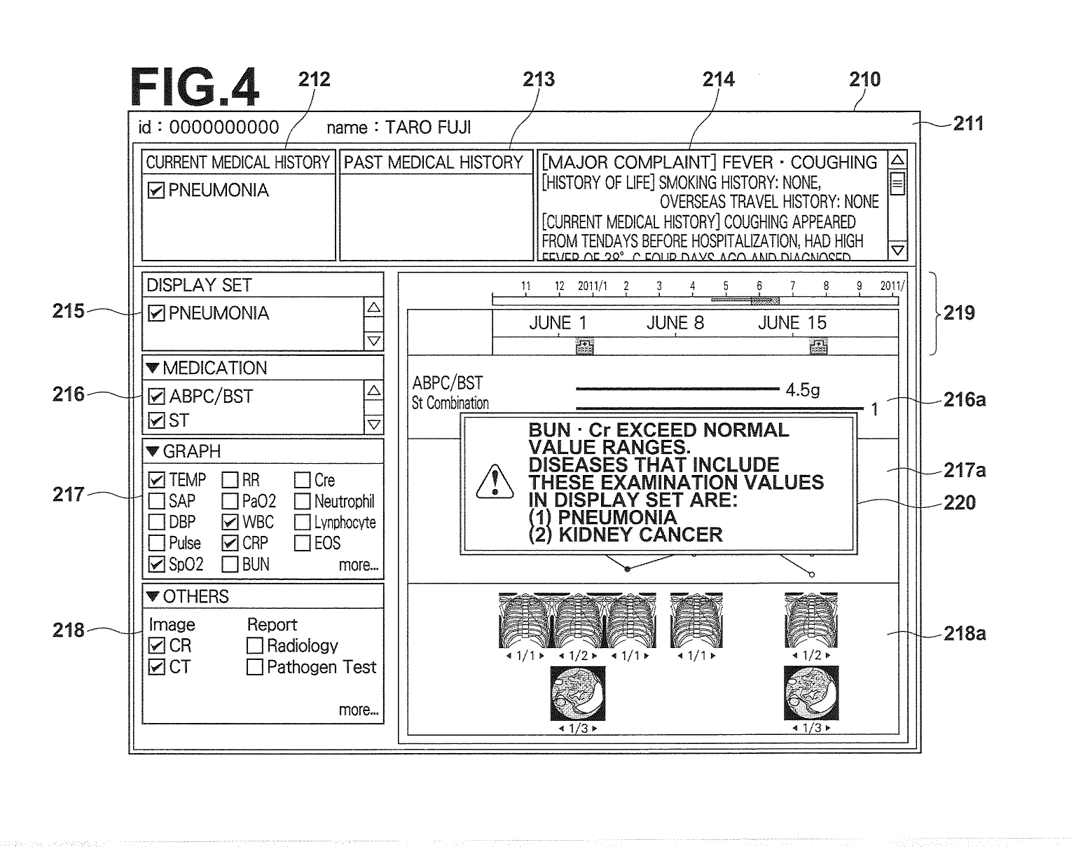

FIG. 4 shows another example of display screen displayed by the display control unit 24. In the display screen of FIG. 4, a warning popup 220 notifying that anomalies are detected in BUN•Cr, which are non-display information, and disease information (disease name list) corresponding to these clinical information items is displayed on the display window 210 in which clinical information corresponding to pneumonia specified as a possible disease of a display target patient is displayed.

For the display screen displaying disease information (disease names or the like) corresponding to the clinical information item in which an anomaly has been detected, as illustrated in FIG. 4, if a user input for selecting any of the displayed diseases is received by the input receiving unit 21, the display control unit 24 has a function to switch according to the user input the current display screen displayed on the display unit 25 to a display screen displaying clinical information of the clinical information item associated with the disease selected by the user input. The display switching is performed by extracting clinical information of the item corresponding to the newly selected disease from the clinical information of the same patient obtained by the clinical information obtaining unit 22 based on the display filter 32 and displaying the extracted clinical information on the display unit 25 according to a preset display option.

The restriction mode setting unit 26 sets a restriction mode which restricts the clinical information items for which an anomaly notification is made at the display control unit 24 by receiving a user input through the input receiving unit 21. For example, a switching button for receiving an instruction to switch between display and non-display may be provided on the display screen and whether to inform or not may be set according to the user input with respect to the switching button. Further, an arrangement may be adopted in which a user input for selecting a clinical information item (or clinical information item group) for which a notification is to be made if an anomaly is detected is received by the input receiving unit 21 and a notification will be made only for the selected clinical information item (clinical information item not restricted).

According to the clinical information system 10 of the present embodiment, a plurality of clinical information items is classified into display and non-display targets, and when clinical information corresponding to the display target clinical information item is displayed on the display unit, an anomaly is detected in clinical information corresponding to a non-display target clinical information item and, if an anomaly is detected, a notification to that effect is given by the configuration described above. This may alert the user of the presence of non-display clinical information that may give a sign of complications or other diseases, thereby preventing overlooking.

In the embodiment described above, the display control unit 24 may have a function to switch between a mode in which all pieces of clinical information obtained for the display target patient are displayed and a mode in which only a piece of clinical information corresponding to the specified disease is displayed according to user input received by the input receiving unit 21.

Further, the display control unit 24 may have a function to display an unread flag when unread information is present in the clinical information obtained for the display target patient.

Still further, in the embodiment described above, a description has been made of a case in which clinical information items are classified into display and non-display targets according to the disease, but they may be classified based on other classification criteria, such as medication information and the like, for display and non-display.

The present invention is not limited to the present embodiment and a portion or all of the components of the clinical information display apparatus may be configured by one computer or by one or more systems, such as computers and the like, connected via a network.

What is claimed is:

1. A clinical information display apparatus in which a plurality of clinical information items is classified into display and non-display targets and clinical information corresponding to a clinical information item classified as the display target is displayed on a display unit, the apparatus comprising:
an anomaly detection unit for detecting an anomaly in clinical information corresponding to a clinical information item classified as the non-display target; and
a notification unit for, when an anomaly is detected by the anomaly detection unit, making a notification to that effect.

2. The clinical information display apparatus of claim 1, wherein the notification unit displays, when an anomaly is detected by the anomaly detection unit, information with respect to the clinical information in which the anomaly has been detected on the display unit in addition to making the notification.

3. The clinical information display apparatus of claim 1, wherein:
the apparatus further comprises a correspondence relationship storage unit storing a correspondence relationship between each clinical information item and a disease associated with the item; and
the notification unit identifies, when an anomaly is detected by the anomaly detection unit, a disease corresponding to the clinical information item of the clinical information in which the anomaly has been detected from the correspondence relationship stored in the correspondence relationship storage unit and displays information with respect to the disease on the display unit in addition to making the notification.

4. The clinical information display apparatus of claim 1, wherein:
the apparatus comprises a mode setting unit for setting a restriction mode that restricts clinical information items for which the notification is made by the notification unit; and
the notification unit makes, when the restriction mode is set by the restriction mode setting unit, the notification only for clinical information items not restricted.

5. The clinical information display apparatus of claim 3, wherein the apparatus comprises:
an input receiving unit for receiving a user input for selecting any disease whose information is displayed on the display screen by the notification unit; and
a display control unit for, when a user input for selecting a disease is received by the input receiving unit, switching the display screen of the display unit to a display screen in which clinical information corresponding to the clinical information item associated in advance with the selected disease is displayed.

6. The clinical information display apparatus of claim 1, wherein the anomaly detection unit detects an anomaly in clinical information constituted by a series of numerical data based on a slope of a graph generated by the clinical information.

7. The clinical information display apparatus of claim 1, wherein the anomaly detection detects an anomaly in clinical information constituted by a series of numerical data obtained over a plurality of times based on whether or not the state in which the numerical data constituting the clinical information are outside of a predetermined criterion value range has continued not less than a predetermined period of time.

8. A clinical information display method performed by a clinical information display apparatus which includes an anomaly detection unit and a notification unit, wherein, when a plurality of clinical information items is classified into display and non-display targets and clinical information corresponding to a clinical information item classified as the display target is displayed on a display unit, the anomaly detection unit detects an anomaly in clinical information corresponding to a clinical information item classified as the non-display target and when an anomaly is detected, the notification unit makes a notification to that effect.

9. The clinical information display method of claim 8, wherein, when an anomaly is detected, the notification unit displays information with respect to the clinical information in which the anomaly has been detected on the display unit in addition to making the notification.

10. A non-transitory computer readable medium on which is recorded a clinical information display program for causing a computer to function as a clinical information display apparatus in which a plurality of clinical information items is classified into display and non-display targets and clinical information corresponding to a clinical information item classified as the display target is displayed on a display unit, wherein the clinical information display apparatus comprises:
an anomaly detection unit for detecting an anomaly in clinical information corresponding to a clinical information item classified as the non-display target; and
a notification unit for, when an anomaly is detected by the anomaly detection unit, making a notification to that effect.
